(19) United States
(12) Patent Application Publication
(10) Pub. No.: US 2011/0135728 A1
(43) Pub. Date: Jun. 9, 2011

(54) GASTRIC RETENTIVE PHARMACEUTICAL COMPOSITIONS FOR EXTENDED RELEASE OF POLYPEPTIDES

(76) Inventors: Jennifer L. Miller, Menlo Park, CA (US); Ryan Douglas Fell, San Carlos, CA (US); Verne Earle Cowles, Dublin, CA (US)

(21) Appl. No.: 12/963,520

(22) Filed: Dec. 8, 2010

Related U.S. Application Data
(60) Provisional application No. 61/267,669, filed on Dec. 8, 2009.

(51) Int. Cl. A61K 9/14; A61K 38/54; A61P 1/18

(52) U.S. Cl. 424/484; 424/94.21

(57) ABSTRACT

Gastric retentive dosage forms for controlled release of polypeptides are described. Methods of treatment using the dosage forms and methods of making the dosage forms are also described.

### CROSS-REFERENCE TO RELATED APPLICATIONS

This application claims priority under 35 U.S.C. §119(e)(1) to U.S. Provisional Application Ser. No. 61/267,669, filed Dec. 8, 2009, is incorporated herein by reference in its entirety.

### TECHNICAL HELD

The present subject matter relates to a dosage form for controlled release of polypeptides to the stomach and small intestine of a patient in the fed mode and to methods of treatment using the dosage forms.

### BACKGROUND

Oral delivery of therapeutic peptide or polypeptide compositions presents great challenges, due in part to the susceptibility of these proteins to degradation in the gastrointestinal tract. Accordingly, much research is being done to design optimal ways to protect the proteins after oral administration but prior to their release and localization to the environment of use.

Currently, oral dosage forms for the delivery of digestive enzymes to the small intestine have been developed and are approved and marketed. These include CREON® (Solvay), PANCREAZE™ (Ortho-McNeil Janssen), and ZENPEP® (Eurand). These products are useful in treating disorders which lead to insufficient digestive enzyme secretion from the pancreas. The lack of digestive enzymes in the upper small intestine leads to malabsorption of nutrients which leads to malnutrition. Some of the most common ailments leading to pancreatic enzyme insufficiency (PEI) are cystic fibrosis, chronic alcoholism, diabetes, chronic pancreatitis, pancreatic surgery, and pancreatic cancer. Delivery of digestive enzymes to the small intestine with each meal aids in the digestion of fats, proteins and carbohydrates, facilitating nutrient absorption.

Oral drug products which deliver digestive enzymes to the small intestine are typically in the form of a capsule containing multiple enteric coated beads or pellets, each bead or pellet containing lipase (breaks down fats), amylase (breaks down carbohydrates) and protease (breaks down proteins). Upon release from the capsules, the beads or pellets empty from the stomach into the small intestine. The digestive enzymes are susceptible to degradation by the acidic conditions of the stomach, and therefore the beads or pellets containing the enzymes are typically coated with an enteric coat which begins to dissolve after the beads or pellets enter into the more basic pH of the small intestine.

These pancreatic enzyme replacement therapy (PERT) products are marketed based on their lipase activity, with several strengths typically available. The enzymes are titrated against symptoms (diarrhea, steatorrhoea, flatulence, nausea) per patient. Titrated dose is typically based on patient weight, meal contents, and/or disease status, with the goal of maintenance of weight (or weight gain, depending on age), and acceptable bowel control.

There remains a compelling need for improvement of these protein delivery products. Previous studies indicate that the size of particles in the stomach is directly correlated with their emptying from the stomach, through the pylorus, and into the duodenum. The smaller the particles the faster their emptying from the fed stomach (Meyer et al., 1988. Gastroenterology, 94:1315-1325; Meyer et al., 1985. Gastroenterology. 89:805-813). As the existing products are small extruded beads that are released from a typical pancreatic enzyme replacement product capsule empty with the liquid fraction of the meal, ahead of the majority of the solid stomach contents. The result is that a majority of the beads are emptied from the stomach ahead of the majority of the meal, leading to incomplete digestion of the meal (Taylor et al., 1999. Arch. Dis. Child., 80:149-152). Taylor et. al. report of “hot spots” of beads in the GI tract, further indicating rapid emptying from the stomach and incomplete mixture of the enzymes with the digestive chyme. The same publication reports: “Patients should spread out their pancreatin (pancrelipase) dosage throughout the meal to limit partitioning and to optimize mixing.”

Accordingly, it would be advantageous to provide an oral dosage form, which when administered with a meal, provides controlled and extended release of polypeptides into the stomach followed by immediate passage of the polypeptides into the small intestine. In the case of pancreatic enzyme release from a gastric retentive dosage form, a dosage form as described herein provides extended release of pancreatic enzymes from the gastric retained dosage form, such as to allow the necessary passage of the enzymes into the small intestine throughout the time period during which chyme enters the small intestine. The technologies described for sustained delivery of pancreatic enzymes to the stomach may be applied to polypeptides in general.

### BRIEF SUMMARY

The present disclosure provides, inter alia, oral gastric retentive dosage forms for oral administration of polypeptides to a subject. The gastric retentive dosage form comprises a swellable hydrophilic matrix in which a polypeptide component is dispersed. Upon oral administration with food, and subsequent imbibition of fluid, the hydrophilic matrix swells to a size sufficient for gastric retention and the polypeptide component is released from the matrix via diffusion and/or erosion.

In one aspect, an oral gastric retentive dosage form comprising a polymeric matrix and a polypeptide component dispersed in the matrix, wherein the matrix comprises at least one hydrophilic polymer and wherein the matrix, upon imbibition of fluid, swells to a size sufficient for gastric retention is provided.

In one embodiment, the dosage form comprises about 5 weight percent (wt %) to about 95 wt % of the polypeptide component. In another embodiment, the dosage form comprises about 10 wt % to about 85 wt %, about 20 wt % to about 75 wt %, about 30 wt % to about 65 wt %, about 40 wt % to about 55 wt %, about 25 wt % to about 50 wt %, about 35 wt % to about 45 wt %, or about 40 wt % to about 45 wt % of the polypeptide component. In still another embodiment, the dosage form comprises about 5 wt %, 10 wt %, 15 wt %, 25 wt %, 30 wt %, 35 wt %, 40 wt %, 45 wt %, 50 wt %, 55 wt %, 65 wt %, 75 wt %, 85 wt %, or 95 wt % of the polypeptide component.

In one embodiment, the polypeptide component comprises a plurality of micropellets, wherein each of the plurality of micropellets comprises one or more polypeptides. In another embodiment, the one or more polypeptides comprises an enzyme. In still another embodiment, the enzyme is a pancreatic enzyme. In yet another embodiment, the pancreatic enzyme is amylase, lipase and/or protease. In another embodiment, each of the plurality of micropellets comprises one or more excipients.

In one embodiment, the enzymes within the plurality of micropellets maintain at least about 70%, 75%, 80%, 85%, 90%, 95%, 96%, 97%, 98% or 99% of their activity when the plurality of micropeilets is exposed to gastric fluid or simulated gastric fluid.

In one embodiment, each of the plurality of micropellets comprises an enteric coating. In another embodiment, the enteric coating of at least one of the plurality of micropellets remains intact until the micropellet reaches the small intestine. In still another embodiment, the enteric coating of at least about 70%, 75%, 80%, 85%, 90%, 95%, 96%, 97%, 98% or 99% of the plurality of micropellets remains intact until the micropellet reaches the small intestine. In yet another embodiment, the intactness of the micropellet enteric coating is measured in terms of activity of the encased one or more enzymes.

In one embodiment, the polypeptide component comprises a plurality of polypeptide particles, wherein each of the plurality of polypeptide particles comprises one or more polypeptides. In still embodiment, the one or more polypeptides comprises an enzyme. In yet another embodiment, the enzyme is an acid-labile enzyme. In another embodiment, the one or more polypeptides comprises a pancreatic enzyme. In yet another embodiment, the pancreatic enzyme is amylase, lipase and/or protease.

In one embodiment, the dosage form comprises one more more hydrophilic polymers having an average molecular weight ranging from about 200,000 Da (Daltons) to about 10,000,000 Da, about 900,000 Da to about 7,000,000 Da, about 2,000,000 Da to about 5,000,000 Da, from about 4,000,000 Da to about 5,000,000 Da, from about 2,000,000 Da to about 4,000,000 Da, from about 900,000 Da to about 5,000,000 Da, or from about 900,000 Da to about 4,000,000 Da. In another embodiment, the dosage form comprises a hydrophilic polymer having an average molecular weight of equal to or greater than about 200,000 Da, 600,000 Da, 900,000 Daltons, 1,000,000 Da, 2,000,000 Da, 4,000,000 Da, 5,000,000 Da, 7,000,000 Da, or 10,000,000 Da.

In one embodiment, the dosage form comprises a hydrophilic polymer having an average viscosity ranging from about 4,000 cp (centipoise) to about 200,000 cp, from about 50,000 cp to about 200,000 op, or from about 80,000 cp to about 120,000 cp, as measured as a 2% weight per volume in water at 20° C.

In one embodiment, the dosage form comprises a total amount of hydrophilic polymer which is between about 40 mg and 800 mg (milligrams) or about 125 mg to about 300 mg. In another embodiment, the total amount of hydrophilic polymer in the dosage form is about 40 mg, 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 ma, 425 mg, 450 mg, 475 mg, 500 mg, 525 mg, 550 mg, 575 mg, 600 mg, 650 mg, 700 mg, 750 mg, or 800 mg. In yet another embodiment, the total amount of hydrophilic polymer in the dosage form is present in an amount which is about 50 wt % to about 40 wt %, about 20 wt % to about 40 wt % or about 10 wt % to about 30 wt % (weight percent) of the dosage form. In yet another embodiment, the total amount of hydrophilic polymer in the dosage form is present in an amount which is about 15 wt %, 17 wt %, 18 wt %, 20 wt %, 22 wt %, 24 wt %, 25 wt %, 27 wt %, 30 wt %, 35 wt %, 45 wt %, 50 wt % or 55 wt % of the dosage form.

In one embodiment, the at least one hydrophilic polymer in the dosage form is a polyalkylene oxide. In another embodiment, the at least one hydrophilic polymer is poly(ethylene oxide). In yet another embodiment, the at least one hydrophilic polymer in the dosage form is a cellulose. In yet another embodiment, the cellulose is hydroxypropyl methylcellulose. In yet another embodiment, the dosage form comprises two hydrophilic polymers in a ratio of 3:1, 3:1.5, 3:2, 2:1, 2:1.5, 1:1, 1:1.5, 1:2, 1:2.5, or 1:3.

In one embodiment, the total amount of polypeptide in the dosage form is about 15 wt % to about 85 wt %, from about 25 wt % to about 80 wt %, from about 15 wt % to about 85 wt %, from about 45 wt % to about 65 wt %, about 15 wt %, about 25 wt %, about 35 wt %, about 45 wt %, about 55 wt %, about 56 wt %, about 75 or about 85 wt %. In another embodiment, the polypeptide is pancreatin or pancrelipase. In yet another embodiment, the polypeptide is an isolated recombinantly produced polypeptide.

In one embodiment, the polypeptide component is released from the dosage form over a time period of 2 h to 12 h (hours), 3 h to 6 h, 2 h to 4 h, 3 h to 7 h, 5 h to 8 h, or 2 h to 8 h. In another embodiment, at least about 80%, 85%, 90%, 95%, 96%, 97%, 98%, or 99% of the polypeptide is delivered to the small intestine of the subject over a time period of at least 1 h, 2 h, 3 h, 4 h, 5 h, 6 h, 7 h, 8 h, 9 h, 10 h, 11 h, or 12 h.

In one embodiment, the polypeptide component is released from the dosage form via diffusion. In another embodiment, the polypeptide component is released from the dosage form via erosion. In yet another embodiment, the polypeptide component is released from the dosage form via a combination of diffusion and erosion.

In one embodiment, the dosage form further comprises one or more binders. In another embodiment, the one or more binders is in an amount ranging from about 15 mg to about 80 mg. In another embodiment, the amount of the one or more binder in the dosage form is about 2.5 wt %, 2.7 wt %, 3.0 wt %, 3.2 wt %, 3.5 wt %, 3.7 wt %, 4.0 wt %, 4.3 wt %, 4.5 wt %, 4.7 wt %, 5.0 wt %, 5.3 wt %, 5.5 wt %, 5.7 wt %, 6.0 wt %, 6.5 wt %, 7.0 wt %, 7.5 wt %, 8.0 wt %, 8.5 wt %, 9.0 wt %, 9.5 wt %, or 10.0 wt % of the dosage form.

In one embodiment, the dosage form comprises a binder which is polyvinylpyrrolidone, hydroxypropylcellulose (HPC), microcrystalline cellulose (MCC), polyvinylalcohol, ethyl cellulose, lactose, or polyethylene glycol. In yet another embodiment, the polyvinylpyrrolidone is povidone, copovidone, or crospovidone. In yet another embodiment, the dosage form comprises a combination of more than one binder.

In one embodiment, the dosage form further comprises a lubricant which is magnesium stearate, calcium stearate, sodium stearyl fumarate, stearic acid, stearyl behenate, glyceryl behenate, or polyethylene glycol.

In one embodiment, the dosage form comprises one or more lubricants which is present in an amount ranging from about 0.3 mg to 10 mg. In yet another embodiment, the amount of the one or more lubricants in the dosage form is about 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 ma, 5.5 mg, 6.0 mg, 6.5 mg, 7.0 mg, 7.5 mg, 8.0 mg, 8.5 mg, 9.0 mg, 9.5 mg or 10 mg. In yet another embodiment, the amount of the one or more lubricants in the dosage form is about 0.4 wt %, 0.5 wt %, 0.6 wt %, 0.7 wt %, 0.8 wt %, 0.9 wt %, 1.0 wt %, 1.2 wt %, 1.4 wt %, 1.6 wt %, 1.8 wt %, 2.0 wt %, 2.2 wt %, 2.4 wt %, or 2.5 wt % of the dosage form.

In one embodiment, the dosage form comprises an anti-oxidant which is ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisuifate, sorbic acid, potassium ascorbate, vitamin E, 4-chloro-2,6-ditertiarybutylphenol, alphatocopherol, or propylgallate. In another embodiment, the antioxidant is present in the dosage form at a wt % (weight percent) of approximately 0.01 wt %, 0.05 wt %, 0.1 wt %, 0.5 wt %, 0.75 wt %, 1 wt %, 2 wt %, 3 wt % or 4 wt %.

In one embodiment, the dosage form comprises one or more additional excipients which are diluents, coloring agents, flavoring agents, and/or glidants.

In one embodiment, the dosage form further comprises an IR layer which comprises an additional active agent. In another embodiment, the additional active agent is between about 5 mg to about 400 mg or between about 10 mg to about 15 mg additional active agent. In another embodiment, the IR layer comprises about 5 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg of the additional active agent. In yet another embodiment, the IR layer comprises an additional active agent which is present in an amount which is about 5 wt % to about 15 wt % of the IR layer. In yet another embodiment, the additional active agent is present in an amount which is about 5 wt %, 6 wt %, 7 wt %, 8 wt %, 9 wt %, 10 wt %, 11 wt %, 12 wt %, 13 wt %, 14 wt %, or 15 wt % of the IR layer.

In one embodiment, the IR layer further comprises a binder. In another embodiment, the binder is polyvinylpyrrolidone (PVP), polyvinylalcohol, hydroxypropyl cellulose (HPC), microcrystalline cellulose (MCC) ethyl cellulose, or polyethylene glycol. In yet another embodiment, the polyvinylpyrrolidone is povidone, copovidone, or crospovidone. In yet another embodiment, the IR layer comprises a combination of more than one binder.

In one embodiment, the IR layer may comprise a binder in an amount ranging from about 6 mg to about 50 mg or from about 6 mg to about 12 mg. In another embodiment, the total amount of the binder in the IR layer is about 5 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 15.0 mg, 17.0 mg, 19.0 mg, 20.0 mg, 23.0 mg, 25.0 mg, 27.0 mg, 30.0 mg, 33.0 mg, 35.0 mg, 37.0 mg, 40.0 mg, 45.0 mg, 47.0 mg, 50.0 mg, 55.0 mg, 57.0 mg, or 60.0 ma. In yet another embodiment, the amount of binder in the IR layer is in an amount which is about 2.5 wt %, 2.7 wt %, 3.0 wt %, 3.2 wt %, 3.5 wt %, 3.7 wt %, 4.0 wt %, 4.3 wt %, 4.5 wt %, 4.7 wt %, 5.0 wt %, 5.3 wt %, 5.5 wt %, 5.7 wt %, 6.0 wt %, 6.5 wt %, 7.0 wt %, 7.5 wt %, 8.0 wt %, 8.5 wt %, 9.0 wt %, 9.5 wt %, or 10.0 wt % of the IR layer.

In one embodiment, the IR layer comprises a disintegrant which is cellulose or a derivative of cellulose such as microcrystalline cellulose, crosspovidone, crosslinked starch such as sodium starch glycolate, alginic acid or soy polysaccharides.

In one embodiment, the IR layer comprises a lubricant which is magnesium stearate, calcium stearate, sodium stearyl fumarate, stearic acid, stearyl behenate, glyceryl behenate, or polyethylene glycol.

In one embodiment, the IR layer comprises an anti-oxidant which is ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiarybutylphenol, alphatocopherol, or propylgallate. In another embodiment, the antioxidant is present in the IR layer at a wt % (weight percent) of approximately 0.01 wt %, 0.05 wt %, 0.1 wt %, 0.5 wt %, 0.75 wt %, 1 wt %, 2 wt %, 3 wt % or 4 wt %.

In one embodiment, the IR layer comprises a lubricant which is present in an amount ranging from about 0.2 mg to about 10.0 mg or from about 1.0 mg to about 10.0 mg. In yet another embodiment, the amount of lubricant in the IR layer is about 0.2 mg, 0.3 mg, 0.4 mg, 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, 6.0 mg, 6.5 mg, 7.0 mg, 75 mg, 8.0 mg, 8.5 mg, 9.0 mg, 9.5 or 10 mg. In yet another embodiment, the amount of lubricant in the IR layer is about 0.4 wt %, 0.5 wt %, 0.6 wt %, 0.7 wt %, 0.8 wt %, 0.9 wt %, 1.0 wt %, 1.2 wt %, 1.4 wt %, 1.6 wt %, 1.8 wt %; 2.0 wt %, 2.1 wt %, 2.2 wt %, 2.4 wt %, or 2.5 wt % of the IR layer.

In one embodiment, the gastric retentive dosage is a tablet. In another embodiment, the tablet has a total weight ranging from about 200 mg to about 1300 mg, or from about 400 mg to about 1000 mg or from about 500 mg to about 800 mg. In yet another embodiment, the tablet has a total weight of about 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg.

In one embodiment, the dosage form is a pharmaceutical tablet, such as a gastric retentive tablet for the extended release of a polypeptide component. In another embodiment, the tablet is a monolithic tablet comprising a polypeptide component dispersed in a polymer matrix, wherein the polymer matrix comprises at least one hydrophilic polymer and wherein the polymer matrix swells upon imbibition of fluid to a size sufficient for gastric retention in a stomach in the fed mode. In another embodiment, the tablet is a monolithic tablet further comprising an IR layer. In another embodiment, the tablet is a bilayer tablet, comprising an extended release (ER) layer and an IR layer. The bilayer tablet is typically a monolithic tablet. In another embodiment, the dosage form is a capsule comprising an ER layer. In another embodiment, the dosage form is a capsule comprising ER layer and an IR lay.

In one embodiment, the tablet has a friability of no greater than about 0.1%, 0.2% 0.3%, 0.4%, 0.5%, 0.7% or 1.0%.

In one embodiment; the tablet has a hardness of at least about 10 kilopond (also known as kilopons) (kp). In another embodiment, the tablet has a hardness of about 7 kp to about 25 kp, or about 12 kp to about 20 kp. In yet another embodiment, the tablet has a hardness of about 11 kp, 12 kp, 13 kp, 14 kp, 15 kp, 16 kp, 17 kp, 18 kp, 19 kp, 20 kp, 21 kp, 22 kp, 23 kp, 24 kg, or 25 kp.

In one embodiment, the tablet has a content uniformity of from about 85 to about 115 percent by weight or from about 90 to about 110 percent by weight, or from about 95 to about 105 percent by weight. In another embodiment, the content uniformity has a relative standard deviation (RSD) equal to or less than about 3.5%, 3.0%, 2.5%, 2.0%, 1.5%, 1.0% or 0.5%.

In one embodiment, the dosage form swells upon imbibition of water from gastric fluid to a size which is approximately 15%, 20%, 30%, 40%, 50%, 75%, 100%, 125%, 150%, 175%, 200%, 225%, 250%, 275%, 300%, 350% or 400% larger than the size of the dosage form prior to imbibition of fluid.

In one embodiment, the dosage form provides a dissolution profile wherein between about 40% to about 80%, about 30% to about 60%, about 35% to about 55% or about 40% to about 50% of the polypeptide component remains in the polymer matrix between about 1 and 2 hours after administration, in one embodiment, not more than 50% of the polypeptide component is released within about the first hour. In a further embodiment, not more than 45% or not more than 40% of the polypeptide component is released within about the first hour. In another embodiment, not more than 85% of the polypeptide component is released within about 4 hours. In yet another embodiment, not less than 50% of the polypeptide component is released after about 6 hours. In yet another embodiment, not less than 60% of the polypeptide component is released after about 6 hours.

In one embodiment, the polypeptide component is released over a time period of less than about 4 hours, less than about 5 hours, less than about 6 hours, less than about 7 hours, or less than about 8 hours in vitro. In another embodiment, the polypeptide component is released over a time period of about 1 hours, 2 hours, 3 hours, 4 hours, 5 hours, 6 hours, 7 hours or 8 hours in vitro.

In one aspect, a method of making a gastric retentive dosage form comprising the polypeptide component dispersed in the polymer matrix comprising at least one hydrophilic polymer is provided.

In one embodiment, the method of making the dosage form comprises granulating the polypeptide component with at least one hydrophilic polymer. In another embodiment, the granulating is fluid bed or high shear granulation. In another embodiment, the method comprises direct compression of the at least one hydrophilic polymer with a pregranulated polypeptide composition. In yet another embodiment, the granulated polypeptide composition contains polypeptide granulated with starch or povidone or polyethylene glycol.

In one embodiment, a gastric retentive dosage form comprising a polypeptide is made by dry blending the polypeptide with the hydrophilic polymer and optionally with one or more excipients prior to compressing the blended mixture into a tablet.

In one embodiment, a gastric retentive dosage form comprising a polypeptide and made by the process of directly compressing a pregranulated polypeptide with one or more hydrophilic polymers is provided.

In one embodiment, a gastric retained dosage form comprising a polymer matrix comprising at least one swellable polymer and a polypeptide component is administered to a subject suffering from cystic fibrosis (CF), chronic pancreatitis, pancreatic exocrine insufficiency, diabetes type I, diabetes type II, surgery including pancreatico-duodenectomy or Whipple's procedure, with or without Wirsung duct injection or total pancreatectomy), obstruction due to pancreatic and biliary duct lithiasis, pancreatic and duodenal neoplasms and ductal stenosis, and other pancreatic disease such as hereditary or post-traumatic and allograft pancreatitis, hemochromatosis, Shwachman's Syndrome, lipamatosis, and hyperparathyroidism

In one embodiment, a gastric retained dosage form is administered to a subject in a fed mode. In another embodiment, the dosage form is administered with a meal to a subject.

### BRIEF DESCRIPTION OF THE DRAWINGS

FIG. 1 is a graph showing cumulative release of beads from a gastric retentive dosage form.

FIG. 2 are graphs showing enzyme activity of beads released from a gastric retentive dosage form.

### DETAILED DESCRIPTION

The various aspects and embodiments will now be fully described herein. These aspects and embodiments may, however, be embodied in many different forms and should not be construed as limiting; rather, these embodiments are provided so the disclosure will be thorough and complete, and will fully convey the scope of the present subject matter to those skilled in the art.

All publications, patents and patent applications cited herein, whether supra or infra, are hereby incorporated by reference in their entirety.

### I. Definitions

It must be noted that, as used in this specification, the singular forms “a,” “an,” and “the” include plural referents unless the context clearly dictates otherwise.

“Optional” or “optionally,” as used herein, means that the subsequently described element, component or circumstance may or may not occur, so that the description includes instances where the element, component, or circumstance occurs and instances where it does not.

The terms “subject,” “individual” or “patient” are used interchangeably herein and refer to a vertebrate, preferably a mammal. Mammals include, but are not limited to, humans.

The term “about,” particularly in reference to a given quantity, is meant to encompass deviations of plus or minus five percent.

The terms “effective amount,” or a “pharmaceutically therapeutically effective amount” a “therapeutically effective amount” refer to the amount of pharmacologically active agent or ingredient to provide the desired effect without toxic effects. The amount of an agent that is “effective” may vary from individual to individual, depending on the age, weight, general condition, and other factors of the individual. An appropriate “effective” amount in any individual may be determined by one of ordinary skill in the art using routine experimentation. An “effective amount” of an agent can refer to an amount that is either therapeutically effective or prophylactically effective or both.

The term “dosage form” refers to the physical formulation of the drug for administration to the patient. Dosage forms include without limitation, tablets, capsules, caplets, liquids, syrups, lotions, lozenges, aerosols, patches, enemas, oils, ointments; pastes, powders for reconstitution, sachets, solutions, sponges, and wipes. Within the context of the present invention, a dosage form will generally be administered to patients in the form of tablets or capsules.

The term “dosage unit” refers to a single unit of the dosage form that is to be administered to the patient. The dosage unit will be typically formulated to include an amount of drug sufficient to achieve a therapeutic effect with a single administration of the dosage unit although where the size of the dosage form is at issue, more than one dosage unit may be necessary to achieve the desired therapeutic effect. For example, a single dosage unit of an active ingredient is typically, one tablet or one capsule. More than one dosage unit may be necessary to administer sufficient drug to achieve a therapeutic effect where the amount of drug causes physical constraints on the size of the dosage form.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. The term “pharmacologically active” (or simply “active”) as in a “pharmacologically active” derivative, refers to a derivative having the same type of pharmacological activity as the parent compound and/or drug and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA, or comparable agency.

The terms “active agent,” “active ingredient,” “therapeutic agent,” and/or “pharmacologically active agent” are used interchangeably herein to refer to any complex or composition or complex that is suitable for oral administration and that has a beneficial biological effect, preferably a therapeutic effect in the treatment or prevention of a disease or abnormal physiological condition. The terms also encompass pharmaceutically acceptable, pharmacologically active derivatives of those active agents specifically mentioned herein, including, but not limited to, conjugates, salts, esters, amides, prodrugs, active metabolites, analogs, and the like. When the terms “active agent,” “pharmacologically active agent,” and “active ingredient” are used, then, or when a particular active ingredient is specifically identified, it is to be understood that applicants intend to include the active ingredient per se as well as pharmaceutically acceptable derivatives.

“Gastric fluid” as used herein encompasses any liquid media which may be used to simulate physiologic gastric fluid. The simulated gastric fluid may be formulated to simulate gastric fluid from a stomach in a fed mode, not in a fed mode or any other physiological state, including that from a diseased or healthy subject.

The terms “sustained release,” “controlled release,” and “extended release” are used interchangeably herein to refer to a dosage form that provides for release of an active ingredient over an extended period of time. With extended release dosage forms, the rate of release of the active ingredient from the dosage form is reduced in order to maintain therapeutic activity of the active ingredient for a longer period of time or to reduce any toxic effects associated with a particular dosing of the active ingredient. Extended release dosage forms have the advantage of providing patients with a dosing regimen that allows for less frequent dosing, thus enhancing compliance. Extended release dosage forms can also reduce peak-related side effects associated with some active ingredients and can maintain therapeutic concentrations throughout the dosing period thus avoiding periods of insufficient therapeutic plasma concentrations between doses.

“Delayed release” dosage forms are a category of modified release dosage forms in which the release of the drug is delayed after oral administration for a finite period of time after which release of the drug is unhindered. Delayed release dosage forms are frequently used to protect an acid-labile drug from the low pH of the stomach or where appropriate to target the GI tract for local effect while minimizing systemic exposure. Enteric coating is frequently used to manufacture delayed release dosage forms.

The term “modified release” refers to a dosage form that includes both delayed and extended release pharmaceutical products. The manufacture of delayed, extended, and modified release dosage forms are known to ordinary skill in the art and include the formulation of the dosage forms with excipients or combinations of excipients necessary to produce the desired active agent release profile for the dosage form.

The “gastric retentive” oral dosage forms described herein are a type of extended release dosage form. Gastric retentive dosage forms are beneficial for the delivery of pharmaceutical agents to the lower GI tract or for local treatment of diseases of the stomach or upper GI tract. For example, in certain embodiments of gastric retentive oral dosage forms of the present invention, the dosage form swells in the gastric cavity and is retained in the gastric cavity of a patient in the fed med so that the drug may be released for heightened therapeutic effect. See, Hou et al., Crit. Rev. Ther. Drug Carrier Syst. 20(6):459-497 (2003).

The term “fed mode,” as used herein, refers to a state which is typically induced in a patient by the presence of food in the stomach, the food-giving rise to two signals, one that is said to stem from stomach distension and the other a chemical signal based on food in the stomach. It has been determined that once the fed mode has been induced, larger particles are retained in the stomach for a longer period of time than smaller particles; thus, the fed mode is typically induced in a patient by the presence of food in the stomach. The fed mode is initiated by nutritive materials entering the stomach upon the ingestion of food. Initiation is accompanied by a rapid and profound change in the motor pattern of the upper GI tract, over a period of 30 seconds to one minute. The change is observed almost simultaneously at all sites along the GI tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 6 hours. Administration of a dosage form “with a meal,” as used herein, refers to administration before, during or after a meal, and more particularly refers to administration of a dosage form about 1, 2, 3, 4, 5, 10, 15 minutes before commencement of a meal, during the meal, or about 1, 2, 3, 4, 5, 10, 15 minutes after completion of a meal.

A “release rate,” as used herein, refers to the quantity of active ingredient released from a dosage form or pharmaceutical composition per unit time, e.g., units of activity detected in dissolution or disintegration media at distinct time points after exposure of the dosage form to fluid or gastric fluid. Release rates for drug dosage forms are typically measured as an in vitro rate of dissolution, i.e., a quantity of active ingredient released from the dosage form or pharmaceutical composition per unit time measured under appropriate conditions and in a suitable fluid. The specific results of dissolution tests claimed herein are performed on dosage forms or pharmaceutical compositions in a USP Type II apparatus and immersed in 0.1 N Hydrochloric acid (HCl) equilibrated in a constant temperature water bath at 37° C. Alternatively, the dosage forms may be immersed in simulated intestinal fluid. Suitable aliquots of the release rate solutions are tested to determine the amount of active ingredient released from the dosage form or pharmaceutical composition. For example, the active ingredient can be assayed or injected into a chromatographic system to quantify the amounts of active ingredient released during the testing intervals.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer” as used herein refers to a molecule containing a plurality of covalently attached monomer units, and includes branched, dendrimeric, and star polymers as well as linear polymers. The term also includes both homopolymers and copolymers, e.g., random copolymers, block copolymers and graft copolymers, as well as uncrosslinked polymers and slightly to moderately to substantially crosslinked polymers, as well as two or more interpenetrating cross-linked networks.

The term “swellable polymer,” as used herein, refers to a polymer that will imbibe a fluid, preferably water, and become enlarged or engorged. A polymer is swellable due, at least in part, to a structural feature of the polymer. Whether or not a swellable polymer when incorporated into a dosage form or matrix containing other components swells in the presence of fluid will depend upon a variety of factors, including the specific type of polymer and the percentage of that polymer in a particular formulation. For example, the term “polyethylene oxide” or “PEO” refers to a polyethylene oxide polymer that has a wide range of molecular weights. PEO is a linear polymer of unsubstituted ethylene oxide and has a wide range of viscosity-average molecular weights. Examples of commercially available PEOs and their approximate molecular weights are: POLYOX® NF, grade WSR coagulant, approximate molecular weight 5 million Da, POLYOX® grade WSR 301, approximate molecular weight 4 million Da. POLYOX® grade WSR 303, approximate molecular weight 7 million Da, POLYOX® grade WSR N-60K, approximate molecular weight 2 million Da, and POLYOX® grade N-80K, approximate molecular weight 200,000 Da. An oral dosage form which comprises a swellable polymer as used herein intends that the polymer when incorporated into the dosage form will swell upon imbibition of water or fluid from gastric fluid.

The terms “swellable” and “bioerodible” (or simply “erodible”) are used to refer to the polymers used in the present dosage forms, with “swellable” polymers being those that are capable of absorbing water and physically swelling as a result, with the extent to which a polymer can swell being determined by the molecular weight or degree of crosslinking (for crosslinked polymers), and “bioerodible” or “erodible” polymers referring to polymers that slowly dissolve and/or gradually hydrolyze in an aqueous fluid, and/or that physically disentangle or undergo chemical degradation of the chains themselves, as a result of movement within the stomach or GI tract.

“Polypeptide particle,” as used herein, means an individual unit of material arising from the purification, or other peptide production processing, of the polypeptide, or that of post-production processing. Polypeptide particle may include products resulting from coalescing processes, including granulation, extrusion, or precipitation processes.

The terms “polypeptide,” “peptide,” and “protein” are used interchangeably herein and refer to a polymer of two or more amino acids joined to each other by peptide bonds or modified peptide bonds, i.e., peptide isosteres. As used herein, the terms encompass amino acid chains of any length. The terms apply to amino acid polymers containing naturally occurring amino acids as well as amino acid polymers in which one or more amino acid residues is a non-naturally occurring amino acid or a chemical analogue of a naturally occurring amino acid. An amino acid polymer may contain one or more amino acid residues that has been modified by one or more natural processes, such as post-translational processing, and/or one or more amino acid residues that has been modified by one or more chemical modification techniques known in the art. The terms also apply to amino acid polymers in which one or more amino acid residue is an artificial chemical mimetic of a corresponding naturally occurring amino acid.

The term “isolated polypeptide” refers to any polypeptide that (1) is free of at least some proteins with which it would normally be found, (2) is essentially free of other proteins from the same source, e.g., from the same species, (3) is expressed by a cell from a different species, or (4) does not occur in nature.

A “fragment” of a reference polypeptide refers to a contiguous stretch of amino acids from any portion of the reference polypeptide. A fragment may be of any length that is less than the length of the reference polypeptide.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A “variant” of a reference polypeptide refers to a polypeptide having one or more amino acid substitutions, deletions, or insertions relative to the reference polypeptide. In certain embodiments, a variant of a reference polypeptide has an altered post-translational modification site (i.e., a glycosylation site). In certain embodiments, both a reference polypeptide and a variant of a reference polypeptide are specific binding agents. In certain embodiments, both a reference polypeptide and a variant of a reference polypeptide are antibodies.

Variants of a reference polypeptide include, but are not limited to, glycosylation variants. Glycosylation variants include variants in which the number and/or type of glycosylation sites have been altered as compared to the reference polypeptide. In certain embodiments, glycosylation variants of a reference polypeptide comprise a greater or a lesser number of N-linked glycosylation sites than the reference polypeptide. In certain embodiments; an N-linked glycosylation site is characterized by the sequence Asn-X-Ser or Asn-X-Thr, wherein the amino acid residue designated as X may be any amino acid residue except proline. In certain embodiments, glycosylation variants of a reference polypeptide comprise a rearrangement of N-linked carbohydrate chains wherein one or more N-linked glycosylation sites (typically those that are naturally occurring) are eliminated and one or more new N-linked sites are created.

Variants of a reference polypeptide include, but are not limited to, cysteine variants. In certain embodiments, cysteine variants include variants in which one or more cysteine residues of the reference polypeptide are replaced by one or more non-cysteine residues; and/or one or more non-cysteine residues of the reference polypeptide are replaced by one or more cysteine residues. Cysteine variants may be useful, in certain embodiments, when a particular polypeptide must be refolded into a biologically active conformation, e.g., after the isolation of insoluble inclusion bodies. In certain embodiments, cysteine variants of a reference polypeptide have fewer cysteine residues than the reference polypeptide. In certain embodiments, cysteine variants of a reference polypeptide have an even number of cysteines to minimize interactions resulting from unpaired cysteines. In certain embodiments, cysteine variants have more cysteine residues than the native protein.

A “derivative” of a reference polypeptide refers to: a polypeptide: (1) having one or more modifications of one or more amino acid residues of the reference polypeptide; and/or (2) in which one or more peptidyl linkages has been replaced with one or more non-peptidyl linkages; and/or (3) in which the N-terminus and/or the C-terminus has been modified. Certain exemplary modifications include, but are not limited to, acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. In certain embodiments, both a reference polypeptide and a derivative of a reference polypeptide are specific binding agents. In certain embodiments, both a reference polypeptide and a derivative of a reference polypeptide are antibodies.

“Conservatively modified variants,” as used herein refers to peptides, polypeptides, or proteins in which individual substitutions, deletions or additions alter, add or delete a single amino acid or a small percentage of amino acids in the peptide, polypeptide or protein sequence, where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V): 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3.sup.rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains (RING, ligase), extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a ligase or RING domain. Typical domains are made up of sections of lesser organization such as stretches of β-sheet and d-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

An “acid-labile enzyme,” as used herein, refers to an enzyme which loses at least 50%, 60%, 70% or 80% of its activity when incubated for at least 1 minute in a pH of about 4.0 or less.

The term “friability,” as used herein, refers to the ease with which a tablet will break or fracture. The test for friability is a standard test known to one skilled in the art. Friability is measured under standardized conditions by weighing out a certain number of tablets (generally 20 tablets or less), placing them in a rotating Plexiglas drum in which they are lifted during replicate revolutions by a radial lever, and then dropped approximately 8 inches. After replicate revolutions (typically 100 revolutions at 25 rpm), the tablets are reweighed and the percentage of formulation abraded or chipped is calculated. The friability of the tablets, of the present invention, is preferably in the range of about 0% to 3%, and values about 1%, or less, are considered acceptable for most drug and food tablet contexts. Friability which approaches 0% is particularly preferred.

The term “tap density” or “tapped density,” as used herein, refers to a measure of the density of a powder. The tapped density of a pharmaceutical powder is determined using a tapped density tester, which is set to tap the powder at a fixed impact force and frequency. Tapped density by the USP method is determined by a linear progression of the number of taps.

The term “bulk density,” as used herein, refers to a property of powders and is defined as the mass of many particles of the material divided by the total volume they occupy. The total volume includes particle volume, inter-particle void volume and internal pore volume.

The term “capping,” as used herein, refers to the partial or complete separation of top or bottom crowns of the tablet main body. For multilayer tablets, capping refers to separation of a portion of an individual layer within the multilayer tablet. Unintended separation of layers within a multilayer tablet prior to administration is referred to herein as “splitting.”

The term “content uniformity,” as used herein refers to the testing of compressed tablets to provide an assessment of how uniformly the micronized or submicron active ingredient is dispersed in the powder mixture. Content uniformity is measured by use of USP Method (General Chapters, Uniformity of Dosage Forms), unless otherwise indicated. A plurality refers to five, ten or more tablet compositions.

“Preventing,” in reference to a disorder or unwanted physiological event in a patient, refers specifically to inhibiting or reducing the occurrence of symptoms associated with the disorder and/or the underlying cause of the symptoms.

“Treating,” “treat,” and “treatment” refer to reduction in severity and/or frequency of symptoms, elimination of symptoms and/or underlying cause, prevention of the occurrence of symptoms and/or their underlying cause, and improvement or remediation of damage.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the present disclosure or its claims.

### II. Gastric Retentive Dosage Form for the Extended Release of Polypeptides

The pharmaceutical compositions described herein, i.e., gastric retained dosage forms comprising polypeptides dispersed in a hydrophilic polymer matrix, provide extended or sustained release of the polypeptides into the stomach. Upon release into the stomach, the polypeptides immediately pass through to the small intestine. The presently described dosage form provides for extended release of polypeptides into the stomach wherein the dosage form is comprised of a polymer matrix that swells upon imbibition of fluid to a size sufficient for gastric retention in the fed mode. Thus, in formulating the dosage forms, properties which simultaneously allow: a) an extent of swelling to provide gastric retention over an extended period, and b) a rate of swelling and erosion that allows release of the polypeptides over a time period of approximately 2 hours to 12 hours, 3 hours to 10 hours, or 4 hours to 6 hours, are provided.

The formulation of these pharmaceutical oral dosage forms preferably result in final products that meet the requirements of regulatory agencies such as the Food and Drug Administration. For example, final dosage forms are preferably stable such that they do not fracture during storage and transport. This is measured for tablets, in part, in terms of friability and hardness. Dosage forms preferably also meet requirements for content uniformity, such that dispersion of the active ingredient(s) is uniform throughout the mixture used to make the dosage form, such that the composition of tablets formed from a particular formulation does not vary significantly from one tablet to another. The FDA requires a content uniformity within a range of 95% to 105%.

The formulation of these pharmaceutical dosage forms must also provide optimal stability of the polypeptides present within the dosage forms.

The dosage form as described here is capable of swelling dimensionally unrestrained in the stomach upon contact with gastric fluid due to the hydrophilic polymer(s) component, such as, polyethylene oxide and/or hypromellose (also known as hydroxypropyl methylcellulose or HPMC, available as METHOCEL®. The Dow Chemical Company), in the formulation, and increase to a size sufficient to be retained in the stomach in a fed mode.

Water-swellable, erodible polymers suitable for use herein are those that swell in a dimensionally unrestrained manner upon contact with water, and gradually erode over time. Examples of such polymers include polyalkylene oxides, such as polyethylene glycols, particularly high molecular weight polyethylene glycols; cellulose polymers such as hydroxyl propyl alkyl celluloses and their derivatives including, but not limited to, alkylcellulose, hydroxyalkyl celluloses, hydroxymethyl cellulose, hydroxyethyl cellulose, hydroxypropyl cellulose (HPC), hydroxypropylmethyl cellulose (HPMC), carboxymethylcellulose (CMC), microcrystalline cellulose; polysaccharides and their derivatives; chitosan; poly(vinyl alcohol); natural or synthetic gums such as guar gum, carrageenan, pectin, locust bean gum xanthan gum, modified starch, and alginates (e.g., calcium alginate), as well as ethyl cellulose, polyvinyl pyrrolidone, fats, waxes, polycarboxylic acids or esters such as Carbopol™, (Noveon IP Holdings Corp.) sweries of polymers, methacrylic acid copolymers, methacryalte polymers (e.g., poly(2-hydroxyethyl methacrylate), poly(acrylic) acid, poly(methacrylic) acid); maleic anhydride copolymers; poly(vinyl pyrrolidone); starch and starch-based polymers; maltodextrins; poly(2-ethyl-2-oxazoline); poly(ethyleneimine); polyurethane; hydrogels; crosslinked polyacrylic acids; and combinations or blends of any of the foregoing. Additional synthetic and/or semisynthetic polymers include, e.g., cellulose acetate phthalate (CAP), polyvinyl acetate phthalate (PVAP), hydroxypropyl methylcellulose phthalate, and/or acrylic polymers, such as methacrylic acid ester copolymers, zein, and the like.

Further examples are copolymers, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA.

Preferred swellable, erodible hydrophilic polymers suitable for forming the gastric retentive portion of the dosage forms described herein are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 9×10⁵ Da to about 8×10⁶ Da. A preferred molecular weight poly(ethylene oxide) polymer is about 5×10⁶ Daltons and is commercially available from The Dow Chemical Company (Midland, Mich.) referred to as SENTRY® POLYOX® water-soluble resins, NE (National Formulary) grade WSR Coagulant. The viscosity of a 1% water solution of the polymer at 25° C. preferably ranges from 4500 to 7500 centipoise.

Dosage forms prepared for oral administration according to the present disclosure will generally contain other inactive additives (excipients) such as binders, lubricants, disintegrants, plasticizers, fillers, stabilizers, surfactants, coloring agents, and the like.

Binders are used to impart cohesive qualities to a tablet, and thus ensure that the tablet or tablet layer remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Examples of polyvinylpyrrolidone include povidone, copovidone and crospovidone.

The dosage form may contain in the IR layer, the ER layer, or both layers, an anti-oxidant for increased stability of the active ingredient as well as the dosage form as a whole. The anti-oxidant may be selected from ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium rnetabisulfate, sorbic acid, potassium ascorbate, vitamin E, 4-chloro-2,6-ditertiarybutylphenol, alphatocopherol, and propylgallate.

The dosage form may contain in the IR layer, the ER layer, or both layers, a chelating agent. Chelating agents tend to form complexes with trace amount of heavy metal ions inactivating their catalytic activity in the oxidation of medicaments. Ethylenediamine tetracetic acid (EDTA) and its salts, dihydroxy ethyl glycine, citric acid and tartaric acid are most commonly used chelators.

Lubricants are used to facilitate tablet manufacture, promoting powder flow and preventing particle capping (i.e. particle breakage) when pressure is relieved. Useful lubricants are magnesium stearate (in a concentration of from 0.25 wt % to 3 wt %, preferably 0.2 w % to 1.0 wt %, more preferably about 0.3 wt %), calcium stearate, stearic acid, and hydrogenated vegetable oil (preferably comprised of hydrogenated and refined triglycerides of stearic and palmitic acids at about 1 wt % to 5 wt %, most preferably less than about 2 wt %). Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

The gastric retentive dosage form may be a single layer, bilayer, or multilayer tablet or it may be a capsule. The tablet comprises a gastric retentive layer comprised of polypeptide dispersed in a matrix of at least one hydrophilic polymer which swells upon imbibition of fluid.

### III. Producing Polypeptides for Extended Release

Polypeptides for formulating the dosage forms as described herein are produced using methods known to those with ordinary skill in the art.

The description below relates primarily to production of polypeptides by culturing cells transformed or transfected with a vector containing nucleic acid encoding the polypeptide of interest. Host cells are transfected or transformed with expression or cloning vectors for polypeptide production and cultured in conventional nutrient media modified as appropriate for inducing promoters, selecting transformants, or amplifying the genes encoding the desired sequences. In general, principles, protocols, and practical techniques for maximizing the productivity of cell cultures can be found in Mammalian Cell Biotechnology: a Practical Approach, M. Butler, ed. (IRL Press, 1991) and Sambrook et al. Molecular Cloning: A Laboratory Manual (New York: Cold Spring Harbor Laboratory Press, 1989). It is, of course, contemplated that alternative methods, which are well known in the art, may be employed to prepare polypeptides. For instance, the polypeptide sequence, or portions thereof, may be produced by direct peptide synthesis using solid-phase techniques (see e.g., Stewart et al., Solid-Phase Peptide Synthesis, W. H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the polypeptide may be chemically synthesized separately and combined using chemical or enzymatic methods to produce the full-length polypeptide.

DNA encoding a polypeptide may be obtained from a cDNA library prepared from tissue believed to possess the polypeptide mRNA and to express it at a detectable level. Accordingly, human polypeptide DNA can be conveniently obtained from a cDNA library prepared from human tissue. The polypeptide-encoding gene may also be obtained from a genomic library or by known synthetic procedures (e.g., automated nucleic acid synthesis), using standard procedures, such as described in Sambrook et al., Molecular Cloning: A Laboratory Manual (New York: Cold Spring Harbor Laboratory Press, 1989). An alternative means to isolate the gene encoding a polypeptide is to use PCR methodology [Sambrook et al., supra; Dieffenbach et al., PCR Primer: A Laboratory Manor (Cold Spring Harbor Laboratory Press, 1995)].

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. In addition to prokaryotes, eukaryotic microbes such as filamentous fungi or yeast are suitable cloning or expression hosts for PRO-encoding vectors. Suitable host cells for the expression of glycosylated PRO are derived from multicellular organisms. Examples of invertebrate cells include insect cells such as Drosophila S2 and Spodoptera Sf9, as well as plant cells. The selection of the appropriate host cell is deemed to be within the skill in the art.

The polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the polypeptide-encoding DNA that is inserted into the vector. The signal sequence may be a prokaryotic signal sequence selected, for example, from the group of the alkaline phosphatase, penicillinase, Ipp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,162), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Still other methods, vectors, and host cells suitable for adaptation to the synthesis of PRO in recombinant vertebrate cell culture are described in Gething et al., Nature, 293:620-625 (1981); Mantei et al., Nature, 281:40-46 (1979); EP 117,060; and EP 117.058.

### IV. Pancreatic Enzymes for Formulation of Oral Gastric Retentive Dosage Forms

In some embodiments, a gastric retentive oral dosage form for the extended release of pancreatic enzymes is provided. Pancreatic enzymes, including but not limited to amylase, lipase and trypsin, can be recombinantly produced as described above, or isolated from the pancreas of warm-blooded mammals.

Pancreatin is a pancreas-derived product that is prepared by drying and hydrolyzing swine pancreas, and contains at least the enzymes amylase, lipase and protease. Pancreatin is made of dried, defatted pancreas. It is prepared from fresh or fresh-frozen pancreas. Normally the pancreas glands are minced and comminuted with the duodenum, which is added to activate the proteolytic enzymes or zymogens in the pancreas. Alternatively, proteolytic activity is sometimes established in the pancreatin preparation by the addition of active trypsin. The blend then undergoes activation of the enzymes. Thereafter, the pancreas is degreased and dried, generally by vacuum drying at room temperature. Pancreatin has been used in the animal industry primarily to treat digestive disturbances. See, e.g., U.S. Pat. No. 5,112,624; see also Russian Patent No. 829,115.

Pancrelipase also is derived from the pancreas of a mammal, and contains amylase, lipase and trypsin. Pancrelipase contains more active lipase enzyme than does pancreatin.

Methods for preparing pancrelipase or pancreatin from the pancreas of warm-blooded animals are well-known in the art and are described, for example, in U.S. Pat. Nos. 3,956,483; 5,674,532; 5,861,177; 5,861,291; 5,993,806; and 7,153,504.

### V. Micropellets Comprising Polypeptides

In some embodiments, a gastric retentive oral dosage form is provided which comprises a swellable hydrophilic matrix in which a plurality of micropellets are dispersed. Each of the plurality of micropellets comprises one or more distinct polypeptides, for example, amylase, lipase, and/or trypsin. Micropellets comprising polypeptides can be prepared by a manufacturing process readily known to one of ordinary skill in the art. See, for example, U.S. Pat. Nos. 4,808,413, 5,225,202, 5,378,462; 7,431,986; and U.S. Patent Publication No. 2007/0148153. In one example, the process comprises the steps of (a) preparing an extrudable mixture comprising about 10% to about 95% pancreatin, about 5% to about 90% of at least one pharmaceutically acceptable binding agent, optionally about 0.01% to about 85% of at least one pharmaceutically acceptable excipient, and water, or one or more enzyme-friendly organic solvents in an amount sufficient to form an extrudabie mixture; wherein the percentages of components are weight to weight of the pancreatin micropellets; (b) creating pancreatin micropellets from the extrudable mixture; (c) forming the pancreatin micropellets into approximately spherical or approximately ellipsoidal shape in the presence of additional enzyme-friendly organic solvent; and (d) removing the water, or one or more enzyme-friendly organic solvents from the pancreatin micropellets such that the pancreatin micropellets are substantially free of the one or more enzyme-friendly organic solvents. Process variations wherein the pancreatin micropellets are substantially free of synthetic oils are preferred.

### VI. Particles Comprising Polypeptides

In some embodiments, a gastric retentive oral dosage form is provided which comprises a swellable hydrophilic matrix in which a plurality of particles are dispersed. The plurality of particles comprise one or more distinct polypeptides, for example, amylase, lipase, and/or trypsin. In this embodiment, the polypeptides may be isolated from a native source or recombinantly produced as described above.

### VII. Enteric Coatings

As described above, the oral gastric retained dosage forms comprise polypeptide components dispersed within a swellable hydrophilic polymer matrix. Upon administration, the dosage form imbibes water and swells to a size sufficient for retention of the dosage form in the stomach in a fed mode. The polypeptide components are then released over a time period of about 2 hours to 4 hours, about 3 hours to about 12 hours, about 1 hour, about 2 hours, about 3 hours, about 4 hours, about 5 hours, about 6 hours, about 7 hours or about 8 hours. The polypeptide components may be either a plurality of particles comprised of polypeptides (polypeptide particles) or a plurality of micropellets comprising polypeptides. The polypeptide particles and the micropellets are each of a size such that they pass through the pylorus to the small intestine essentially immediately upon release from the dosage form.

In some embodiments, the polypeptide particles and micropeliets comprise an enteric coating. Enteric coatings will remain intact in the stomach but will rapidly dissolve once they arrive at the small intestine, thereafter releasing the drug at sites downstream in the intestine (e.g., the ileum and colon). Enteric coatings are well known in the art and are discussed at, for example, Remington's Pharmaceutical Sciences, Mack Publishing Co., Easton, Pa.; and Polymers for Controlled Drug Delivery, Chapter 3, CRC Press, 1991. Methods for applying enteric coatings to pharmaceutical compositions are well known in the art, and include for example, U.S. Patent Publication No. 2006/0045822.

Enteric coatings have long been used to inhibit release of drug from tablets and pellets, to protect the drug from degradation in the stomach. The enteric coatings are resistant to stomach acid for required periods of time depending on the composition and/or thickness thereof, before they begin to disintegrate and allow for slow release of drug in the stomach and/or upper intestines. Some examples of coatings previously employed are beeswax and glyceryl monostearate; beeswax, shellac and cellulose; and cetyl alcohol, mastic and shellac as well as shellac and stearic acid (U.S. Pat. No. 2,809,918); polyvinylacetate and ethyl cellulose (U.S. Pat. No. 3,835,221); and neutral copolymer of polymethacrylic acid esters (Eudragit L30D), (F. W Goodhart et al, Pharm. Tech., pp 64-71, April, 1984); copolymers of methacrylic acid and methacrylic acid methyl ester (Eudragits), or a neutral copolymer of polymethacrylic acid esters containing metallic stearates (Mehta et al U.S. Pat. Nos. 4,728,512 and 4,794,001), and hyprornellose phthalate. Most available enteric coating polymers begin to become soluble at pH 5.5 and above, with maximum solubility rates at pH's greater than 6.5.

As a means of optimizing the pancrelipase delivery system to best mimic healthy human physiology, it is advantageous to liberate the pancrelipase from the enteric bead as fast as possible after passing through the pylorus into the proximal small intestine. As a means of accelerating the rate of dissolution of the enteric coat, a half-thickness, double coat of enteric material (for instance, Eudragit L30 D-55) may be applied, wherein the inner enteric coat is buffered up to pH 6.0 in the presence of 10% citric acid, followed by a final layer of standard Eudragit L 30 D-55, Applying two layers of enteric coat, each half the thickness of a typical enteric coat, the team of Liu and Basit were able to demonstrate accelerated enteric coating dissolution compared to a similar coating system applied, unbuffered, as a single layer (Liu, F. and Basit, A. Journal of Controlled Release. 147 (2010) 242-245.)

In general, known enteric coatings may exhibit a low flexibility. Due to such a low flexibility, fissuring can occur and the damaged enteric coating film will not provide protection during stomach passage. Further, fissuring or breaking of the enteric coating may also lead to a declined bioavailability. Accordingly, it is important that the enteric coating of the polypeptide-containing micropellets remains intact during the process of manufacturing the gastric retained dosage form.

The process described herein for manufacturing a gastric retained dosage form comprising micropellets dispersed within a polymeric matrix may involve at least dry or wet blending, or other such tableting processes. Exposure of the micropellets would be expected by an ordinary artisan to be subject to breakage or other more subtle structure compromise. Any such breakage would expose the polypeptides contained within the micropellets to the highly acidic environment of the stomach, leading to inactivation and/or degradation of the enzymes before they reach the small intestine, where the enzymatic activity is required.

In some embodiments, the dosage forms described herein comprise a plurality enteric-coated micropellets in which the enteric coating for each of the micropellets remains substantially intact. When the enteric coating for the plurality of micropellets remains substantially intact, this indicates that the polypeptides within at least about 70% of the plurality of micropellets within the dosage form are not exposed to the acidic environment upon ingestion of the dosage by a subject or upon submersion of the dosage form in a solution which simulates gastric fluid. An enteric coating which remains substantially intact may also reflect that at least about 75%, 80%, 85%, 90% or 95% of the polypeptides within each micropellet are not exposed to the acidic environment upon ingestion or other exposure to acidic media.

The intactness of the enteric coating may be determined by measuring, for example, the enzymatic activity of any enzymes which are encased within each of the micropellets. As a specific example, a gastric retentive tablet manufactured as described herein, may be incubated in a simulated gastric fluid, allowing elution of the micropellets from the dosage form. The micropellets can then be assayed to measure the activity of the enzyme(s) contained therein. As described in Example 1, micropellets containing pancrelipase (a combination of lipases, proteases and amylases derived from an animal such as a pig), which have been released from the dosage form in a simulated gastric fluid having an acidic pH, can be assayed for lipase, protease and amylase activity.

Accordingly, the micropellets dispersed in the gastric retentive dosage form may maintain at least 70% of the enzyme activity upon exposure to gastric fluid or simulated gastric fluid. To measure a loss of enzyme activity, a controlled experiment can be performed. For example, a plurality of enteric-coated micropellets are incubated at either a relatively neutral pH (about 6.0-7.5) or at an acidic pH (1.0-5.5). The incubation can be at any constant temperature. A temperature of about 37.5° C. may be used to simulate physiological conditions, but room temperature (e.g., about 20° C. or 25° C.) can be used. The reagents of the relevant assay are then added to the micropellets at a more neutral pH (i.e., a pH which simulates that found in the small intestine), and enzyme activity is measured. If the enzyme activity measured for the micropellets incubated at an acidic pH is less than that for micropellets measured at a more neutral pH (at a pH simulating that of the small intestine), this indicates that the structure of the enteric coating was degraded to some extent during incubation in the acidic fluid.

Methods for assaying enzyme activity are well-known to the skilled artisan. Assays used in Example 1 were derived from the USP monograph for pancrelipase in USP 32-NF 27.

### VIII. Methods to Manufacture Oral Gastric Retentive Dosage Forms

The gastric retentive tablets as disclosed herein may be made by direct compression or by a granulation procedure. Direct compression is used with a group of ingredients can be blended, placed onto a tablet press, and made into a perfect tablet without any of the ingredients having to be changed. Powders that can be blended and compressed are commonly referred to as directly compressible or as direct-blend formulations. When powders do not compress correctly, they must be granulated.

Granulation is a manufacturing process that increases the size and homogeneity of active pharmaceutical ingredients and excipients in a solid dosage formulation. The granulation process, which is often referred to as agglomeration, changes physical characteristics of the dry formulation, with the aim of improving manufacturability, and therefore, product quality.

Granulation technology can be classified into one of two basic types: wet granulation and dry granulation. Wet granulation is the more prevalent agglomeration process utilized within the pharmaceutical industry. Most wet granulation procedures follow some basic steps; the drug(s) and excipients are mixed together, and a binder solution is prepared and added to the powder mixture to form a wet mass. The moist particles are then dried and sized by milling or by screening through a sieve. In some cases, the wet granulation is “wet milled” or sized through screens before the drying step. There are four basic types of wet granulation; high shear granulation, fluid bed granulation, extrusion and spheronization and spray drying.

When incorporating relatively dense beads into a tablet matrix comprising typical powdered excipients, it is sometimes helpful to cogranulated the beads in with some of the excipients, particularly in a fluid bed granulation. An example of such a method is found in U.S. Pub. No. 2009/0028941), incorporated herein by reference.

A. Fluid Bed Granulation

The fluid bed granulation process involves the suspension of particulates within an air stream while a granulation solution is sprayed down onto the fluidized bed. During the process, the particles are gradually wetted as they pass through the spay zone, where they become tacky as a result of the moisture and the presence of binder within the spray solution. These wetted particles come into contact with, and adhere to, other wetted particles resulting in the formation of particles.

A fluid bed granulator consists of a product container into which the dry powders are charged, an expansion chamber which sits directly on top of the product container, a spray gun assembly, which protrudes through the expansion chamber and is directed down onto the product bed, and air handling equipment positioned upstream and downstream from the processing chamber.

The fluidized bed is maintained by a downstream blower that creates negative pressure within the product container/expansion chamber by pulling air through the system. Upstream, the air is “pre-conditioned” to target values for humidity, temperature and dew point, while special product retention screens and filters keep the powder within the fluid bed system.

As the air is drawn through the product retention screen it “lifts” the powder out of the product container and into the expansion chamber. Since the diameter of the expansion chamber is greater than that of the product container, the air velocity becomes lower within the expansion chamber. This design allows for a higher velocity of air to fluidize the powder bed causing the material to enter the spray zone where granulation occurs before loosing velocity and falling back down into the product container. This cycle continues throughout the granulation process.

The fluid bed granulation process can be characterized as having three distinct phases; pre-conditioning, granulation and drying. In the initial phase, the process air is pre-conditioned to achieve target values for temperature and humidity, while by-passing the product container altoaether. Once the optimal conditions are met, the process air is re-directed to flow through the product container, and the process air volume is adjusted to a level that will maintain sufficient fluidization of the powder bed. This pre-conditioning phase completes when the product bed temperature is within the target range specified for the process.

In the next phase of the process, the spraying of the granulating solution begins. The spray rate is set to a fall within a pre-determined range, and the process continues until all of the solution has been sprayed into the batch. It is in this phase where the actual granulation, or agglomeration, takes place.

Once the binder solution is exhausted, the product continues to be fluidized with warm process air until the desired end-point for moisture content is reached. This end-point often correlates well with product bed temperature, therefore in a manufacturing environment, the process can usually be terminated once the target product bed temperature is reached. A typical fluid bed process may require only about thirty to forty-five minutes for the granulation step, plus ten to fifteen minutes on either side for pre-conditioning and drying.

As with any of the wet granulation processes, one variable is the amount of moisture required to achieve successful agglomeration. The fluid bed granulation process requires a “thermodynamic” balance between process air temperature, process air humidity, process air volume and granulation spray rate. While higher process air temperature and process air volume add more heat to the system and remove moisture, more granulating solution and a higher solution spray rate add moisture and remove heat via evaporative cooling. These are the process parameters which must be evaluated as a manufacturing process is developed, and the key is understanding the interdependency of each variable.

Additional factors affecting the outcome of the fluid bed granulation process are the amount and type of binder solution, and the method by which the binder is incorporated within the granulation. Other process variables are the total amount of moisture added through the process, and the rate at which the moisture content is increased. These parameters can have an effect on the quality and the characteristics of the granulation. For instance, a wetter fluid bed granulation process tends to result in a stronger granule with a higher bulk density. However, an overly aggressive process, where moisture is added too rapidly, can loose control over achieving the final particle size and particle size distribution objectives.

B. High Shear Granulation

Many pharmaceutical products manufactured by wet granulation utilize a high shear process, where blending and wet massing are accomplished by the mechanical energy generated by an impeller and a chopper. Mixing, densification and agglomeration are achieved through the “shear” forces exerted by the impeller; hence the process is referred to as high shear granulation.

The process begins by adding the dry powders of the formulation to the high shear granulator, which is a sealed “mixing bowl” with an impellor which rotates through the powder bed, and a chopper blade which breaks up over-agglomerates which can form during the process. There are typically three phases to the high shear process; dry mixing, solution addition, or wet massing and high shear granulation.

In the first phase, dry powders are mixed together by the impeller blade which rotates through the powder bed. The impeller blade is positioned just off the bottom of the product container. There is a similar tolerance between the tips of the impeller blade and the sides of the container. The impeller blades rotation trough the powder bed creates a “roping” vortex of powder movement. The dry mixing phase typically lasts for only a few minutes.

In the second phase of the process, a granulating liquid is added to the sealed product container, usually by use of a peristaltic pump. The solution most often contains a binder with sufficient viscosity to cause the wet massed particles to stick together or agglomerate. It is common for the solution addition phase to last over a period of from three to five minutes. While the impeller is rotating rather slowly during this step of the process, the chopper blade is turning at a fairly high rate of speed, and is positioned within the product container to chop up over-sized agglomerates, while not interfering with the impellers movement.

Once the binder solution has been added to the product container, the final stage of the granulation process begins. In this phase, high shear forces are generated as the impeller blades push through the wet massed powder bed, further distributing the binder and intimately mixing the ingredients contained therein. The impeller and chopper tool continue to rotate until the process is discontinued when the desired granule particle size and density end-points are reached. This end-point is often determined by the power consumption and/or torque on the impeller.

Once the high shear granulation process has been completed, the material is transferred to a fluid bed dryer, or alternatively, spread out onto trays that are then placed in a drying oven, where the product is dried until the desired moisture content is achieved, usually on the order of 1-2% as measured by Loss On Drying (LOD) technique.

A variable that affects the high shear process is the amount of moisture required to achieve a successful granulation. A key to the process is having the right amount of moisture to allow for agglomeration to occur. Too little moisture will result in an under-granulated batch, with weak bonds between particles and smaller, to non-existent particles, with properties similar to those of the dry powder starting materials. On the other hand, excess moisture can result in a “crashed” batch with results varying from severe over-agglomeration to a batch that appears more like soup.

Other formulation parameters affecting the outcome of the high shear granulation process are the amount and type of binder solution, and the method by which the binder is incorporated within the granulation. For example, it is possible to include some of the binder in the dry powder mixture as well as in the granulating solution, or it may be incorporated only in the granulating solution or only in the dry powder, as is the case where water is used as the granulating solution.

The high shear granulation process parameters which are variable include impeller and chopper speeds, the solution addition rate, and the amount of time allocated to the various phases of the process. Of these, preferred variables for consideration are the solution addition rate and the amount of time the wet massed product is under high shear mixing.

C. Extrusion and Spheronization

This specialized wet granulation technique involves multiple processing steps and was developed to produce very uniform, spherical particles ideally suited for multi-particulate drug delivery of delayed and sustained release dosage forms.

Similar to high shear granulation initially, the first step involves the mixing and wet massing of the formulation. Once this step is complete, the wet particles are transferred to an extruder that generates high forces used to press the material out through small holes in the extruder head. The extrudate is of uniform diameter and is then transferred onto a rotating plate for spheronization. The forces generated by the rotating plate initially break up the extruded formulation strands into uniform lengths. Additional dwell time within the spheronizer creates particles that are round and uniform in size. These pellets or spheres are then dried to the target moisture content, usually within a fluid bed system.

Pellets produced in this manner tend to be dense, and have a capacity for high active ingredient loading, approaching 90% or more in some cases. The pellet size is uniform, and the size distribution is narrow, as compared to other granulation approaches. This quality assures consistent surface area within and between batches, which is desired when functional coatings are subsequently applied to create sustained release formulations, delayed release formulations and formulations designed to target a specific area within the body.

Uniform surface area is desired because the pharmaceutical coating process endpoint is determined not by coating thickness, but by the theoretical batch weight gain of the coating material. If the batch surface area is consistent, then the coating thickness will also be consistent for a given weight gain, and coating thickness is the primary variable in determining the functionality of the coating system, whether the goal is controlling the duration of sustained release formulations or imparting an acid resistant characteristic to “beads” necessary to protect certain compounds which would otherwise be severely degraded in the presence of the acidic environment of the stomach.

D. Spray Drying

Spray drying is a unique and specialized process that converts liquids into dry powders. The process involves the spraying of very finely atomized droplets of solution into a “bed” or stream of hot process air or other suitable gas. Not typically utilized for the conventional granulation of dosage form intermediates, spray drying has gained acceptance within the industry as a robust process that can improve drug solubility and bioavailability.

Spray drying can be used to create co-precipitates of a drug/carrier that can have improved dissolution and solubility characteristics. In addition, the process can also be useful as a processing aid. For example, it is much more difficult to maintain the uniformity of a drug in suspension, as compared to the same compound in solution. One may have a need to develop an aqueous coating or drug layering process utilizing a drug that is otherwise not soluble in water. By creating a co-precipitate of the drug and a suitable water soluble carrier, often a low molecular weight polymer, the co-precipitate will remain in solution throughout the manufacturing process, improving uniformity of the spray solution and the dosage form created by the coating process. Uniformity is particularly desired where lower doses of potent compounds are intended to be coated onto beads or tablet cores.

This same process may be used to enhance the solubility and bioavailability of poorly soluble drugs. By complexing certain excipients and the active ingredient within a solvent system which is then spray dried, it is possible to enhance the drugs absorption within the body. Selection of the solvent system, the complexing agent(s) and the ratios utilized within the formulation are formulation variables that influence the effectiveness of solubility enhancement utilizing the spray drying technique. Other process parameters with an effect on drug solubility are the temperatures of the spray solution and process gas, the spray rate and droplet size and the rate of re-crystallization. The spray dried granulations created by these techniques can then be incorporated into capsules or tablets by conventional manufacturing processes.

E. Dry Granulation

The dry granulation process involves three basic steps; the drug(s) and excipients(s) are mixed (along with a suitable binder if needed) and some form of lubrication, the powder mixture is compressed into dry “compacts,” and then the compacts are sized by a milling step. The two methods by which dry granulation can be accomplished are slugging and roller compaction.

### IX. Methods of Making and Characterizing the Extended Release Gastric Retentive Dosage Forms Disclosed Herein

In one aspect, a method of making a gastric retentive extended-release dosage form as a single layer tablet comprising dry blending of the plurality of polypeptide particles or polypeptide minipellets with water-swellable polymers and other excipients is provided. The dry blended mixture is then compressed into tablets.

Extended release polymer matrices comprising particles or minipellets are made using one or more swellable polymers, such as those including, but not limited to, POLYOX® 1105 (approximate molecular weight of 900,000 Daltons), POLYOX® N-60K (approximate molecular weight of 2,000,000 Daltons), or POLYOX® WSR-301 (approximate molecular weight of 4,000,000 Daltons).

Bulk and tap densities for the drug, excipient or blends can be determined as follows. A graduated cylinder is filled with a certain amount of material measured by mass, and the volume recorded to determine the material bulk density. Tap density can be determined with a help of a Tap Density Tester (for instance, the Varian Tapped Density Tester) by exposing the material to 100 taps per test and recording the new volume, Data are reported as mass per volume.

Particle size determination is performed immediately after granulation and drying, after sieving through 20 mesh screen to remove agglomerates. Particle diameter is determined with a sieve-type particle diameter distribution gauge using sieves with openings of 44, 53, 75, 106, 150, and 250 mesh. Fractions are weighed on Mettler balance to estimate size distribution. This provides determination of the quantitative ratio by particle diameter of composition comprising particles. Sieve analysis according to standard United States Pharmacopoeia methods (e.g., USP-23 NF 18), may be done such as by using a Meinzer II Sieve Shaker.

The granulated mixture can be blended with the polymer, filler, cushioning agent and lubricant in a V-blender. The resultant mixture can be compressed into monolithic, single-layer tablets using a Manesty® BB4 press. Tablets may be prepared at a rate, for example, of approximately 800 tablets per minute.

Tablets can then be characterized for disintegration and dissolution release profiles, hardness, friability and content uniformity.

The dissolution profiles for the tablets may be determined gravimetricatly in USP apparatus III (for instance, the Varian BioDis™ III) in pH 1 media (0.1 N HCl) or in modified simulated gastric fluid, 37° C. At each timepoint, for instance, 1, 2, 3, and 4 hours, the dissolution apparatus will move the dosage form from one media tube vessel to next media tube vessel, leaving behind the enteric beads that had eroded out of the dosage form and fell through the mesh at the bottom of the reciprocating sample tubes. The beads left behind in the media tube vessel are then decanted, dried, and their mass recorded. The resulting cumulative gravimetric release profiles for the tablets are based upon the fraction of the theoretical mass of beads added to the formulations that are recovered at each timepoint.

A disintegration tester measures the time it takes a tablet to break apart in solution. The tester suspends tablets in a solution bath for visual monitoring of the disintegration rate. Both the time to disintegration and the disintegration consistency of all tablets are measured. The disintegration profile is determined in a USP Disintegration Tester in pH 5.8 phosphate buffer, Samples, 1 ml at each time-point, may be taken, for example, without media replacement at 0, 5, 1, 2, 3, 4, 5, 6, 7 and 8 hours. The resulting cumulative disintegration profiles are based upon a theoretical percent active added to the formulation is determined.

Tablet hardness changes rapidly after compression as the tablet cools. In the case of the presently disclosed gastric retentive dosage forms, a tablet that is too hard may not be able to imbibe fluid rapidly enough to prevent passage through the pylorus in a stomach in a fed mode. A tablet that is too soft may break apart, not handle well, and can create other defects in manufacturing. A soft tablet may not package well or may not stay together in transit.

After tablets are formed by compression, it is desired that the tablets have a strength of at least 7-25 Kiloponds (Kp), preferably at least about 12-20 (Kp). A hardness tester is used to determine the load required to diametrically break the tablets (crushing strength) into two equal halves. The fracture force may be measured using a Venkel Tablet Hardness Tester, using standard USP protocols.

Friability is a well-known measure of a tablet's resistance to surface abrasion that measures weight loss in percentage after subjecting the tablets to a standardized agitation procedure. Friability properties are especially relevant during any transport of the dosage form as any fracturing of the final dosage form will result in a subject receiving less than the prescribed medication. Friability can be determined using a Roche Friability Drum according to standard USP guidelines that specify the number of samples, the total number of drum revolutions and the drum rpm to be used. Friability values of from 0.8 to 1.0% are regarded as constituting the upper limit of acceptability.

The prepared tablets may be tested for content uniformity to determine if they meet the pharmaceutical requirement of 90-110% of theoretical drug content per tablet. Each tablet is placed in a solution of 1.0 N HCl and stirred at room temperature until all that remains are the enteric particles. The particles are then decanted and dried, and the mass recovered is compared to the theoretical mass added to each tablet and reported as percent drug content. In another aspect, a method of making a bilayer tablet comprising a gastric retentive extended-release layer and an immediate release layer is provided.

V. Methods for Treating Conditions

In another aspect, a subject suffering from conditions including, but not limited to cystic fibrosis (CF), chronic pancreatitis, pancreatic exocrine insufficiency, diabetes type I, diabetes type H, surgery including pancreatico-duodenectomy or Whipple's procedure, with or without Wirsung duct injection or total pancreatectomy), obstruction due to pancreatic and biliary duct lithiasis, pancreatic and duodenal neoplasms and ductal stenosis, and other pancreatic disease such as hereditary or post-traumatic and allograft pancreatitis, hemochromatosis, Shwachman's Syndrome, lipomatosis, and hyperparathyroidism, is provided

Generally, the frequency of administration of a particular dosage form is determined to provide the most effective results in an efficient manner without overdosing and varies according to the following criteria: (1) the characteristics of the particular active ingredient(s), including both its pharmacological characteristics and its physical characteristics, such as stability; (2) the rate of release of drug from the dosage form (3) the relative amounts of the active ingredient and polymer, and (4) the individual's response to the therapeutic dose delivered. In the case of pancreatic enzyme replacement therapy, administration of pancrelipase should be optimized towards the optimal reduction of gastrointestinal disruption, specifically, nausea, flatulence, diarrhea, abdominal pain, bulky stools, or steatorrhoea. In most cases, the dosage form is prepared such that effective results are achieved with administration with each meal and snack. Alternatively, the dosage form is prepared to provide efficacy when administered with each meal. Furthermore, the dosage form is prepared to provide efficacy once every three hours, once every six hours, once every eight hours, or once every twelve hours. As previously discussed, due to the physical constraints placed on a tablet or capsule that is to be swallowed by a patient, most dosage forms can only support a limited amount of drug within a single dosage unit.

For all modes of administration, the gastric retentive dosage forms described herein are preferably administered in the fed mode, i.e., with or just after consumption of a meal (see U.S. Publication No, 2003/0104062, herein incorporated by reference).

In some aspects, the postprandial or fed mode can also be induced pharmacologically, by the administration of pharmacological agents that have an effect that is the same or similar to that of a meal. These fed-mode inducing agents may be administered separately or they may be included in the dosage form as an ingredient dispersed in the matrix of the tablet, as a separate layer in a bilayer configuration, or as an overcoat. Examples of pharmacological fed-mode inducing agents are disclosed in U.S. Pat. No. 7,405,238, entitled “Pharmacological Inducement of the Fed Mode for Enhanced Drug Administration to the Stomach,” inventors Markey. Shell, and Berner, the contents of which are incorporated herein by reference.

### EXAMPLES

The following examples illustrate certain aspects and advantages of the present invention, however, the present invention is in no way considered to be limited to the particular embodiments described below.

### Example 1

### Formulation of Prototype I Gastric Retentive Tablets Having Pancreatin Micropellets

Gastric retentive tablets containing CREON 1224 pellets were manufactured and evaluated. This experiment was done to investigate the ability of the dosage form to deliver the pellets over 4 hours. Enzymatic activity provided by the released pellets was assayed following dissolution of the oral dosage form in acidic pH.

CREON 1224 pellets were compressed in a matrix formulation designed to swell upon hydration to a size which would allow gastric retention of the dosage form in a stomach in the fed mode. A blend of excipients, as provided in Table 1, was thoroughly mixed using a dry blend process. An amount of the resultant blend required to make a 1000 mg tablet was weighed out onto a weigh paper. The enteric coated pellet contents of a CREON 1224 capsule was emptied onto the weigh paper with the blend. Using a small spatula, the pellets and excipients were carefully mixed prior to being filled into the die of an 12×16 mm modified oval. The tablets were hand made on a Carver Auto C Press (Fred Carver, Inc., Wabash, Ind.). The resulting mixture was compressed at 1000 lbs. to form the tablet. The settings on the Carver Press were: 0 second dwell time (the setting on the Carver Press), and 100% pump speed.

The formulation for the pancrelipase tablet prototype described is set forth in Table 1 below, The Carbowax 4000 was included as a cushioning agent to limit the damage to the enteric coat on the CREON 1224 pellets. A low molecular weight polyethylene glycol was chosen as to provide limited influence on the erosion of the tablet. The Pearlitol was included to enhance the rate of water penetration into the tablet, which was desirable to achieve a 3-4 hour release target.

TABLE 1

Formula A

| Ingredient | Mg | wt % |
|---|---|---|
| CREON 1224 pellets | 455 | 45.5 |
| POLYOX ® 1105 | 150 | 15.0 |
| CARBOWAX ® 4000 | 300 | 30.0 |
| Pearlitol 300 DC | 90 | 9.0 |
| Magnesium Stearate | 5 | 0.5 |
| Total | 1000.0 | 100.0 |

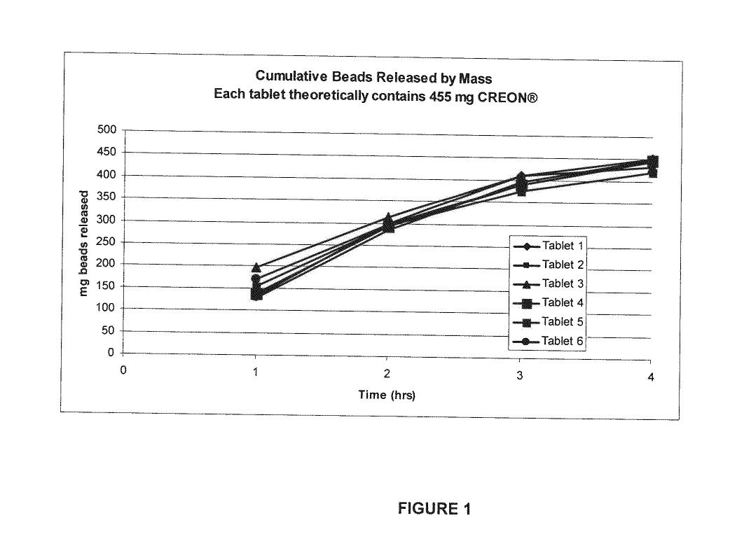

Release rate characteristics of the pancrelipase gastric retentive tablets were investigated using a USP Dissolution Apparatus III containing 250 ml modified 0.1 N HCl, pH 1.0, and 37±0.5° C. with a 20 mesh screen at the top and bottom of the reciprocating cylinder. Sampling was done every hour for 4 hours. The mass of beads released at each time point was measured (see FIG. 1). The results are presented in Table 2 below and graphically shown in FIG. 1.

TABLE 2

| | Lot 09082402-TD-RF | | | | | |
|---|---|---|---|---|---|---|
| Time | Mg Beads Released | | | | | |
| (hours) | Tablet 1 | Tablet 2 | Tablet 3 | Tablet 4 | Tablet 5 | Tablet 6 |
| 1 | 132 | 128 | 196 | 138 | 154 | 171 |
| 2 | 300 | 284 | 314 | 293 | 296 | 297 |
| 3 | 409 | 395 | 410 | 390 | 376 | 390 |
| 4 | 451 | 448 | 433 | 446 | 421 | 445 |

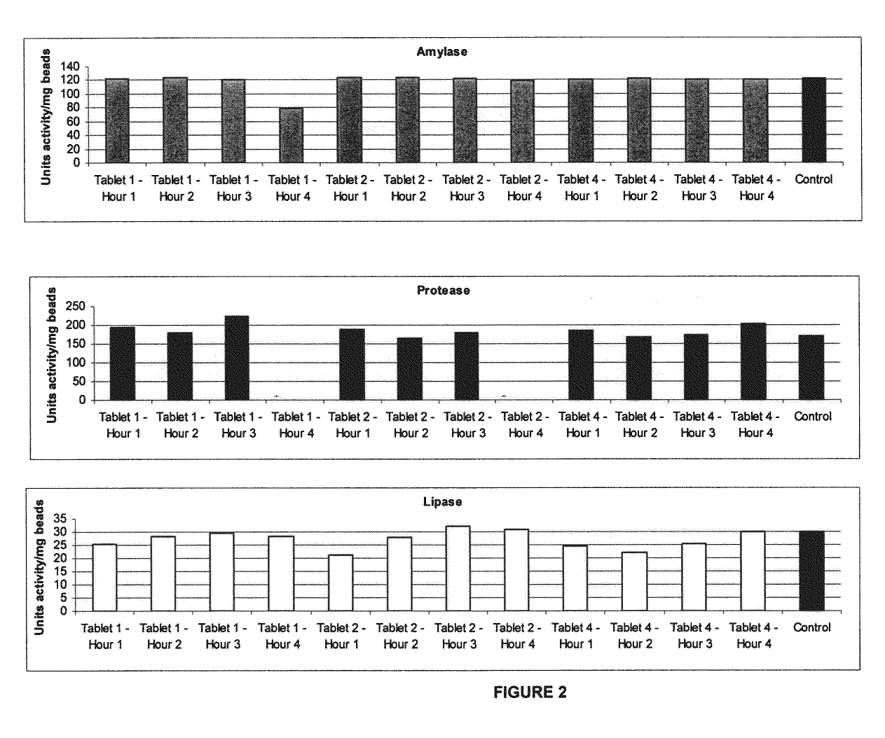

The activity of each of the three enzymes in the released CREON 1224 pellets was then assayed for each time point. The results are presented in Table 3 and shown graphically in FIG. 2. The control was 455 mg uncompressed CREON 1224 pellets. In the Protease 4-hour assay, Tablets 1 and 2, the “na” indicates that not enough beads were recovered to complete the Protease assay. Activity assay for each of the enzymes was measured according to methods listed in the USP monograph for Pancrelipase in USP 32-NF 27, and as described in more detail below.

TABLE 3
GR Prototype II - 4 hr Formula

| | Amylase | Protease | Lipase |
|---|---|---|---|
| | (U/mg) | (U/mg) | (U/mg) |
| Tablet 1 | | | |
| 1 hour | 123 | 194 | 25.5 |
| 2 hour | 124 | 178 | 28.4 |
| 3 hour | 120 | 225 | 29.6 |
| 4 hour | 79 | na | 28.4 |
| Control | 122 | 172 | 30.1 |
| Tablet 2 | | | |
| 1 hour | 124 | 187 | 21.1 |
| 2 hour | 124 | 164 | 28.1 |
| 3 hour | 122 | 179 | 32 |
| 4 hour | 119 | na | 30.7 |
| Control | 122 | 172 | 30.1 |
| Tablet 4 | | | |
| 1 hour | 120 | 184 | 24.7 |
| 2 hour | 123 | 167 | 22.1 |
| 3 hour | 121 | 174 | 25.3 |
| 4 hour | 120 | 203 | 29.8 |
| Control | 122 | 172 | 30.1 |

To measure amylase activity (at 25° C.), a standard preparation solution of USP Pancreatin Amylase and Protease RS (0.2 mg/ml in 200 mM phosphate buffer, pH 6.8) was prepared. Also prepared was an assay preparation solution containing 4 times the amylase activity of the USP Pancreatin Amylase and Protease RS. Into each of 4 containers labeled: S (standard enzyme), U (test enzyme), BS (blank for the standard enzyme), and BU (blank for the test enzyme), a mixture was added which contained: 25 ml of substrate solution (0.2 μl of purified soluble starch dissolved in water), 10 ml 200 mM phosphate buffer, pH 6.8, and 1 ml 200 mM NaCl.

To containers BU and BS, 2 ml of 1 N hydrochloric acid (HCl) was added and mixed. To flasks U and BU, 1.0 ml assay preparation solution was added.

After 10 minutes, 2 ml 1 N HCl was added to flasks S and U, and mixed. To each container, 10.0 ml of 0.1 N iodine VS was added, with continuous stirring, followed by the addition of 45 ml 0.1 N sodium hydroxide (NaOH). The containers were then placed in the dark at a temperature between 15° C. and 25° C. for 15 minutes. To each container, 4 ml of 2 N sulfuric acid was added, and the solution was titrated with 0.1 N sodium thiosulfate VS to the disappearance of the blue color.

The amylase activity was calculated, in USP Units per mg of the Pancrelipase Fancrelipase taken, by the formula:

100(Cs/Wu)*(Vbu−Vu)/(Vbs−Vs),

in which Cs is the amylase activity of the Standard preparation, in USP Units per ml, Wu is the amount, in mg, of pancrelipase taken, and Vu, Vs, Vbu and Vbs are the volumes, in ml, of 0.1 N sodium thiosulfate consumed in the titration of the solutions in flasks U, S, BU, and BS, respectively.

To assay for lipase activity, the following procedure was performed. An olive oil substrate was prepared containing 165 ml acacia (0.1 g/ml), 20 ml olive oil, and 15 g crushed ice. The mixture was cooled in an ice bath to 5° C., and homogenized at high speed for 15 minutes using an electric blender, intermittently cooling in an ice bath to prevent the temperature from exceeding 30° C.

To make the buffer solution, 60 mg of tris(hydroxymethyl)aminomethane and 234 mg of sodium chloride were dissolved in water to make 100 ml. (5 mM tris(hydroxymethyl)aminomethane, 40 mM NaCl)

To prepare a solution of bile salts, 80.0 mg of USP Bile Salts RS (80 mg/ml) were dissolved in each ml of a buffer solution (5 mM tris(hydroxymethyl)aminomethane, 40 mM NaCl).

A standard test dilution of USP Pancreatin Lipase RS was prepared, containing 8-16 USP units of lipase activity per ml. An assay test dilution of Pancrelipase was prepared containing 8-16 USP units of lipase activity per ml.

To perform the assay, 10.0 ml of olive oil substrate, 8.0 ml buffer solution (5 mM tris(hydroxymethyl)aminomethane, 40 mM NaCl), 2.0 ml bile salts solution, and 9.0 ml water was mixed in a jacketed glass vessel of about 50-ml capacity. With the mixture maintained at a temperature of about 37° C., 0.1 N NaOH VS, was added to adjust the pH to 9.20 potentiometrically using a calomel-glass electrode system. 1.0 ml assay test dilution was added, then 0.1 N NaOH VS was added for 5 minutes to maintain the pH at 9.0. The volume of 0.1 N sodium hydroxide VS added after each minute was recorded. In the same manner, 1.0 ml standard test dilution was titrated.

To calculate potency, the volume of 0.1 N NaOH VS titrated was plotted against time. Using only the points which fall on the straight-line segment of the curve, the mean acidity released per minute by the test specimen and the standard. Taking into consideration the dilution factors, the lipase activity was calculated, in USP Units, of the pancrelipase taken by comparison to the activity of the Reference Standard, using the lipase activity stated on the label of USP Pancreatin Lipase RS.

To measure protease activity, a solution of casein (12.5 mg/ml in 0.01 N NaOH, pH 8) was used as the substrate. A standard solution of USP Pancreatin Amylase and Protease (1 mg/ml in 50 mM potassium phosphate buffer, pH 7.5) was mixed to produce a concentration of about 2.5 USP units of protease activity per ml. To generate a standard curve, 3 dilutions of the standard enzyme solution were prepared, which contained 1.25 u, 2 u and 2.5 u of protease activity. To each tube, 25 mg of casein solution was added and the tubes were incubated at 40° C. Sixty minutes after addition of the casein, the reactions were stopped by the addition of trichloroacetic acid to a concentration of about 175 mM. The mixtures were allowed to stand at room temperature for 10 minutes to complete protein precipitation, and were then filtered. Absorbance of the filtrate was measured at 280 nm. The absorbance values for the filtranes were corrected by substracting the absorbance from corresponding control reactions (no casein), and the corrected absorbance values were plotted against the corresponding values.

As shown in the results, there was no significant decrease in enzyme activity for any of the three enzymes following release of the CREON 1224 pellets into the acidic dissolution media. This indicates that enteric coating of the CREON 1224 pellets remained intact throughout the process of tabletting.

TABLE 3
GR Prototype II - 4 hr Formula

| | Amylase | Protease | Lipase |
|---|---|---|---|
| | (U/mg) | (U/mg) | (U/mg) |
| Tablet 1 | | | |
| 1 hour | 123 | 194 | 25.5 |
| 2 hour | 124 | 178 | 28.4 |
| 3 hour | 120 | 225 | 29.6 |
| 4 hour | 79 | 0 | 28.4 |
| Control | 122 | 172 | 30.1 |
| Tablet 2 | | | |
| 1 hour | 124 | 187 | 21.1 |
| 2 hour | 124 | 164 | 28.1 |
| 3 hour | 122 | 179 | 32 |
| 4 hour | 119 | 0 | 30.7 |
| Control | 122 | 172 | 30.1 |
| Tablet 4 | | | |
| 1 hour | 120 | 184 | 24.7 |
| 2 hour | 123 | 167 | 22.1 |
| 3 hour | 121 | 174 | 25.3 |
| 4 hour | 120 | 203 | 29.8 |
| Control | 122 | 172 | 30.1 |

### Example 2

### Potential Patient Benefit of Optimized Mixing of Pancreatic Enzymes and Food

As a means of investigating whether patients would benefit from optimized mixing of pancreatic replacement therapy (i.e. pancrelipase) and food over the course of gastric emptying of a meal, a study is done to compare a gastric retentive oral dosage form, designed to deliver the enzymes throughout the meal, at various doses (as measured by Lipase units) with an existing product, for instance, CREON 1224, which delivers all the enzymes at the beginning of the meal.

Thirty-six Cystic Fibrosis (CF) patients already on existing pancreatic enzyme replacement therapy, aged 7-18 years old, are dosed at a specific dose within the label recommendation of existing therapy, in this case, CREON 1224 (see prescribing information). The patients are randomized into 4 cohorts and administered one of the four therapeutic regimen listed below, and then crossed over such that each patient will receive each therapeutic regimen.

Therapeutic Regimen:
Reference: CREON 1224 dosed at 1000 U/kg body weight/meal with each snack and meal
Formulation 1: GR1: 500 U/kg dosed at each meal (skip snack dose)
Formulation 2: GR2: 1000 U/kg dosed at each meal (skip snack dose)
Formulation 3: 1500 U/kg dosed at each meal (skip snack dose)

Patients will be administered a diet based on recommendations by the Cystic Fibrosis Foundation. Accordingly, patients will be administered 3 high fat meals and snacks per day: a high-fat breakfast, lunch and dinner, with high-fat snacks, at times listed below. Total fat consumption is equal to or greater than 100 grams/day).
Breakfast: 7:00 am.
Snack 1: 10:00 a.m.
Lunch: 1:00 p.m.
Snack 2: 4:00 p.m.
Dinner: 7:00 p.m.
Snack 3: 10:00 p.m.

Each cohort is randomized to one of the four therapeutic regimens. The patients are dosed according to the therapeutic regimen for 6 days and then, switching to the next regimen according to the randomization schedule, each cohort will complete each of the four therapeutic regimens.

Stool samples are collected for the last 72 hours of each treatment. Stool samples are visually assessed as to consistency and number of bowel movements over the 72-hour test period. The samples are assayed for fat content to determine the coefficient of fat absorption (CFA) according to the following formula:

CFA=((fat intake(g)−fat excretion(g))/fat intake(g))×100.

Fecal fat determination is analyzed according to the method described in Van de Kramer, et al. (1949; “Rapid method for determination of fat in feces.” J Biol Chem; 177:347-55).

Proof of pancrelipase delivery concept is investigated by comparing the CFA of existing treatment (the CFA of all cohorts on Reference CREON 1224) administered with all meals and snacks with the CFA of Formulations 1, 2, and 3 administered with meals only.

The patient benefit of optimizing the mixing of food (chyme) and the pancrelipase via a gastric retentive, controlled-release of the enzymes can be measured by observing a similar CFA between the reference CREON 1224 regimen and at least one of test formulations (1, 2 or 3), as less pancrelipase would be delivered for the same result (as measured as units of lipase delivered per day). In one embodiment, the formulation and dosing regimen could be considered successful if it were demonstrated that any one of Formulations 1, 2 or 3 resulted in equal or higher CFA compared to Reference. CREON 1224, thus demonstrating a patient benefit of improved convenience, i.e. only having to take their pancrelipase three times per day with each meal with Formulations 1, 2, or 3, compared with having to take pancrelipase with each meal and snack, i.e. six administrations per day, of the reference CREON 1224.

1. An oral gastric retentive dosage form comprising,
a polymeric matrix wherein the polymeric matrix is comprised of at least one hydrophilic polymer, wherein upon imbibition of fluid the polymeric matrix swells to a size sufficient for gastric retention in a stomach in a fed mode, and
a polypeptide component dispersed in the polymeric matrix.

2. The dosage form of claim 1, wherein the polypeptide component comprises a plurality of micropellets.

3. The dosage form of claim 1, comprising about 5 wt % to about 95 wt % of the polypeptide component.

4. The dosage form of claim 2, wherein each of the plurality of micropellets comprises one or more polypeptides.

5. The dosage form of claim 4, wherein at least one of the one or more polypeptides is an enzyme.

6. The dosage form of claim 5, wherein the enzyme is a pancreatic enzyme.

7. The dosage form of claim 2, wherein each of the plurality of micropellets comprises an enteric coating.

8. The dosage form of claim 7, wherein the enteric coating comprises an inner and an outer layer.

9. The dosage form of claim 6, wherein the enzyme maintains at least about 30% of its activity after exposure of the micropellet to gastrointestinal fluid.

10. The dosage form of claim 1, wherein about 20%-50% of the polypeptide component is released from the dosage form within about 1 hour after immersion in a gastric fluid.

11. The dosage form of claim 1, wherein at least about 80% of the polypeptide component is released from the dosage form within about 8 hours after immersion in a gastric fluid.

12. A method of treating a disease which results in a polypeptide deficiency comprising,
administering to a subject in a fed mode an oral gastric retentive dosage form comprising,
a polymeric matrix wherein the polymeric matrix is comprised of at least one hydrophilic polymer, wherein upon imbibition of fluid the polymeric matrix swells to a size sufficient for gastric retention in a stomach in the fed mode, and
a polypeptide component dispersed in the polymeric matrix.

13. The method according to claim 12, wherein the polypeptide component comprises a plurality of micropellets.

14. The method according to claim 12, wherein the polypeptide component comprises an enzyme.

15. The method according to claim 14, wherein the enzyme is a pancreatic enzyme.
